(19) United States
(12) Patent Application Publication
(10) Pub. No.: US 2007/0112055 A1
(43) Pub. Date: May 17, 2007

(54) Crystalline forms of almotriptan and processes for their preparation

(75) Inventors: Ramasubramanian Sridharan, Navi Mumbai (IN); Vandanapu Loka Appala Purushotham, Navi Mumbai (IN); Kori Sadhana Algooram, Mumbai (IN); Nitin Sharad Chandra Pradhan, Manpada (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/541,879

(22) Filed: Oct. 2, 2006

Related U.S. Application Data
(60) Provisional application No. 60/739,362, filed on Nov. 23, 2005.
(60) Provisional application No. 60/737,198, filed on Nov. 16, 2005.

(30) Foreign Application Priority Data
Oct. 28, 2005 (IN) 1355/MUM/2005
Sep. 30, 2005 (IN) 1229/MUM/2005

(51) Int. Cl. A61K 31/405; C07D 403/02

(52) U.S. Cl. 514/414; 548/465

(57) ABSTRACT

Crystalline forms of almotriptan and almotriptan malate are disclosed. Processes for their preparation and pharmaceutical compositions containing the same are also disclosed. The amorphous form of almotriptan malate, processes for its preparation and a pharmaceutical composition containing the same are also disclosed

### PRIORITY

This application claims the benefit under 35 U.S.C. § 119 to U.S. Provisional Application No. 60/739,362, filed on Nov. 23, 2005, and entitled “CRYSTALLINE ALMOTRIPTAN AND PROCESS FOR ITS PREPARATION”; U.S. Provisional Application No. 60/737,198, filed on Nov. 16, 2005, and entitled “AMORPHOUS ALMOTRIPTAN MALATE”; Indian Provisional Application No. 1355/MUM/2005, filed on Oct. 28, 2005, and entitled “CRYSTALLINE ALMOTRIPTAN AND PROCESS FOR THE PREPARATION THEREOF”; and Indian Provisional Application No. 1229/MUM/2005, filed on Sep. 30, 2005, and entitled “AMORPHOUS ALMOTRIPTAN MALATE AND PROCESS FOR THE PREPARATION THEREOF”, the contents of each of which are incorporated by reference herein.

### BACKGROUND OF THE INVENTION

1. Technical Field

The present invention generally relates to a novel crystalline form of almotriptan base and a process for its preparation. The present invention also generally relates to an amorphous and crystalline form of almotriptan malate and processes for their preparation.

2. Description of the Related Art

Almotriptan, also known as 1-[[[3-[2-(dimethylamino)ethyl]indol-5-yl]methyl]sulfonyl]pyrrolidine), is represented by the structure of Formula I. 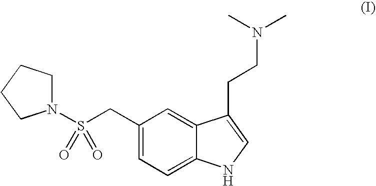
Generally, almotriptan binds with high affinity to 5-HT_{1D}, 5-HT_{1B} and 5-HT_{1F} receptors. Almotriptan has weak affinity for 5-HT_{1A} and 5-HT₇ receptors. The malate salt of almotriptan is indicated for the acute treatment of migraine with or without aura in adults. Almotriptan malate is sold under the trade names AXERT® and ALMOGRAN®. See, e.g., The Merck Index, Thirteenth Edition, 2001, pp. 56, monograph 301; and Physician's Desk Reference, “Micardis,” 58th Edition, pp. 2433-2436 (2004).

Polymorphic forms occur where the same composition of matter crystallizes in a different lattice arrangement resulting in, for example, different thermodynamic properties and stabilities specific to the particular polymorph form. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. It is well known that the crystalline polymorph form of a particular drug is often an important determinant of the drug's ease of preparation, stability, solubility, storage stability, ease of formulation and in vivo pharmacology. Thus, in cases where two or more polymorph substances can be produced, it may be desirable to have a method to make both polymorphs in pure form.

In deciding which polymorph is preferable, the numerous properties of the polymorphs must be compared and the preferred polymorph chosen based on the many physical property variables. It is entirely possible that one polymorph form can be preferable in some circumstances where certain aspects such as ease of preparation, stability, etc are deemed to be critical. In other situations, a different polymorph maybe preferred for greater solubility and/or superior pharmacokinetics. Polymorphic forms of a compound can be distinguished in a laboratory by X-ray diffraction spectroscopy and by other methods such as infrared spectrometry. Additionally, polymorphic forms of the same drug substance or active pharmaceutical ingredient, can be administered by itself or formulated as a drug product (also known as the final or finished dosage form), and are known in the pharmaceutical art to affect, for example, the solubility, stability, flowability, tractability and compressibility of drug substances and the safety and efficacy of drug products.

The amorphous forms in a number of drugs exhibit different dissolution characteristics and in some cases different bioavailability patterns compared to crystalline forms. See, e.g., Konne T., Chem Pharm Bull, 38, 2003 (1990). For some therapeutic indications, one bioavailability pattern may be favored over another. An amorphous form of cefuroxime axietil is an example of one amorphous drug exhibiting much higher bioavailability than the crystalline forms, which leads to the selection of the amorphous form as the final drug substance for cefuroxime axietil pharmaceutical dosage form development. Additionally, the aqueous solubility of crystalline atorvastatin calcium is lower than its amorphous form, which may result in the difference in their in vivo bioavailability. An amorphous form of almotriptan malate has now been discovered.

U.S. Pat. No. 5,565,447 (“the '447 patent”), herein incorporated by reference, discloses almotriptan base. The '447 patent further discloses that almotriptan base was purified by column chromatography (See Example 1) to obtain a white foam.

Because improved drug formulations showing, for example, better bioavailability or better stability are consistently sought, there is an ongoing need for new or purer polymorphic forms of existing drug molecules. The new forms of almotriptan described herein are believed to help meet these and other needs.

### SUMMARY OF THE INVENTION

In accordance with one embodiment of the present invention, crystalline Form G of almotriptan is provided.

In accordance with a second embodiment of the present invention, crystalline Form G of almotriptan characterized by a powder x-ray diffraction (XRD) pattern having characteristic peaks (expressed in degrees 2θ±0.20°θ) at approximately one or more of the following positions: about 13.96, about 14.25, about 15.48, about 21.5 and about 23.7 is provided.

In accordance with a third embodiment of the present invention, crystalline Form G of almotriptan having at least one of the following characteristics is provided:

(a) an XRD pattern substantially in accordance with FIG. 1; and/or

(b) a Differential Scanning Calorimetric (DSC) thermogram substantially in accordance with FIG. 2; and/or

(c) a predominant endotherm peak at about 102.79° C.

In accordance with a fourth embodiment of the present invention, a pharmaceutical composition is provided comprising a therapeutically effective amount of crystalline Form G of almotriptan.

In accordance with a fifth embodiment of the present invention, a pharmaceutical composition is provided comprising a therapeutically effective amount of crystalline Form G of almotriptan characterized by a powder XRD pattern having characteristic peaks (expressed in degrees 2θ±0.2°θ) at approximately one or more of the following positions: about 13.96, about 14.25, about 15.48, about 21.5, and about 23.7.

In accordance with a sixth embodiment of the present invention, a pharmaceutical composition is provided comprising a therapeutically effective amount of crystalline Form G of almotriptan having at least one of the following characteristics:

(a) a XRD pattern substantially in accordance with FIG. 1; and/or

(b) a DSC thermogram substantially in accordance with FIG. 2; and/or

(c) a predominant endotherm peak at about 102.79° C.

In accordance with a seventh embodiment of the present invention, a method for treating a migraine is provided, the method comprising administering to a patient a therapeutically effective amount of crystalline Form G of almotriptan.

In accordance with an eighth embodiment of the present invention, a process for preparing crystalline Form G of almotriptan is provided, the process comprising crystallizing almotriptan free base in one or more solvents.

In accordance with a ninth embodiment of the present invention, an amorphous form of almotriptan malate is provided.

In accordance with a tenth embodiment of the present invention, a process for preparing an amorphous form of almotriptan malate is provided, the process comprising the steps of:

(a) dissolving substantially non-amorphous almotriptan malate in a solvent solution comprising one or more solvents capable of dissolving substantially non-amorphous almotriptan malate; and

(b) recovering an amorphous form of almotriptan malate from the solution.

In accordance with an eleventh embodiment of the present invention, a process for preparing an amorphous form of almotriptan malate is provided, the process comprising the steps of:

(a) reacting almotriptan base with malic acid in a solvent solution comprising an alcohol; and

(b) lyophilizing the solution to provide the amorphous form of almotriptan malate.

In accordance with a twelfth embodiment of the present invention, substantially pure almotriptan malate in an amorphous form is provided.

In accordance with a thirteenth embodiment of the present invention, a pharmaceutical composition is provided comprising a therapeutically effective amount of an amorphous form of almotriptan malate.

In accordance with a fourteenth embodiment of the present invention, a pharmaceutical composition is provided comprising a therapeutically effective amount of substantially pure almotriptan malate in an amorphous form.

In accordance with a fifteenth embodiment of the present invention, a method for treating a migraine in a subject in need of such treatment is provided, which comprises administering to the subject a therapeutically effective amount of an amorphous form of almotriptan malate.

In accordance with a sixteenth embodiment of the present invention, a crystalline form of almotriptan malate is provided having an XRD pattern substantially in accordance with FIG. 4.

In accordance with a seventeenth embodiment of the present invention, a process for preparing a crystalline form of almotriptan malate is provided, the process comprising the steps of:

(a) reacting almotriptan base with malic acid in a solvent solution comprising a first alcohol; and

(b) drying the solution to obtain a residue;

(c) dissolving the residue in a second alcohol under reflux; and

(d) cooling the solution to recover the crystalline form of almotriptan malate from the solution.

In accordance with an eighteenth embodiment of the present invention, a process for preparing a crystalline form of almotriptan malate is provided, the process comprising (a) providing a solution comprising crystalline Form G of almotriptan and malic acid in a solvent; and (b) substantially removing the solvent from the solution to provide the crystalline form of almotriptan malate.

In accordance with a nineteenth embodiment of the present invention, a pharmaceutical composition is provided comprising a therapeutically effective amount of a crystalline form of almotriptan malate having an XRD pattern substantially in accordance with FIG. 4.

### DEFINITIONS

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “subject” or “a patient” or “a host” as used herein refers to mammalian animals, preferably human.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

The term “diluent” or “filler” as used herein is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The termn “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, polyvinylpyrrolidone (PVP), tyloxapol (a nonionic liquid polymer of the alkyl aryl polyether alcohol type, also known as superinone or triton), combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### BRIEF DESCRIPTION OF THE DRAWINGS

FIG. 1 is a characteristic powder XRD pattern of crystalline Form G of almotriptan.

FIG. 2 is a characteristic DSC thermogram of crystalline Form G of almotriptan.

FIG. 3 is a characteristic powder XRD pattern for an amorphous form of almotriptan malate.

FIG. 4 is a characteristic powder XRD of a crystalline form of almotriptan malate.

### DETAILED DESCRIPTION OF THE PREFERRED EMBODIMENTS

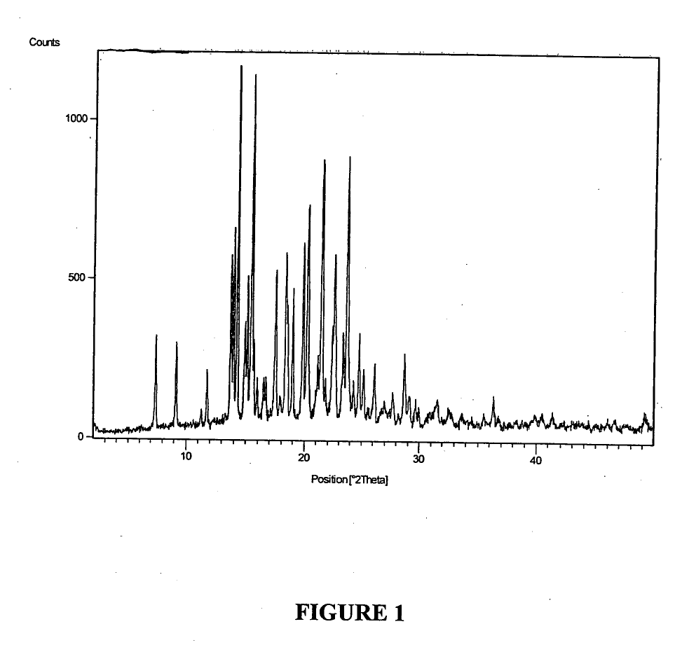

One embodiment of the present invention is directed to a novel polymorph of almotriptan, designated crystalline Form G. Crystallinity may be measured using methods familiar to those skilled in the art. These methods include, but are not limited to powder X-ray diffraction, differential scanning calorimetry, dynamic vapor sorption, isothermal microcalorimetry, inverse gas chromatography, near infra-red spectroscopy and solid-state NMR. The novel polymorph Form G of almotriptan may be characterized by, for example, X-ray powder diffraction pattern and/or melting point. The powder XRD pattern for crystalline Form G of almotriptan of the present invention is presented substantially in accordance with FIG. 1. The x-ray powder diffractogram was measured on a X'Pert Pro PANalytical. The DSC thermogram can be measured using any suitable instrument known in the art. Crystalline Form G of almotriptan has at least one, and preferably all, of the following properties:

(a) an XRD pattern substantially in accordance with FIG. 1; and/or

(b) an XRD pattern exhibiting characteristic peaks (expressed in degrees 2θ±0.2°θ) at approximately one or more of the positions: 7.38 [m], 9.14[m], 11.75[w], 13.74[m], 13.96[s], 14.25[s], 14.91[m], 15.11[m], 15.48[s], 15.64[m], 15.96[w], 16.49[w], 16.67[w], 17.51[m], 18.36[m], 18.49[m], 18.99[m], 19.87[m], 20.25[s], 21.14[w], 21.5[s], 21.8[w], 22.36[m], 22.57[m], 23.31[m], 23.7[s], 24.27[w], 24.76[m], 25.17[w], 26.1[w], and 28.69[w]; wherein (s)=strong intensity; (m)=medium intensity and (w)=weak intensity; and/or

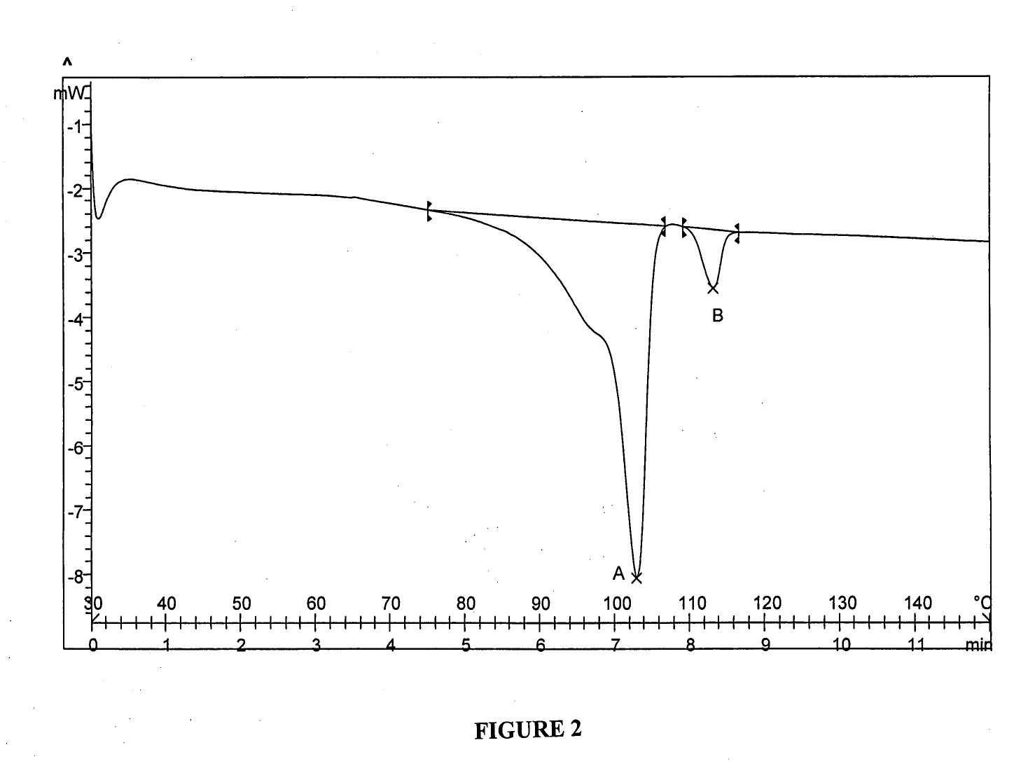

(c) a DSC thermogram substantially in accordance FIG. 2; and/or

(d) a predominant endotherm peak at about 102.79° C. and/or a weak endotherm peak at about 113.24° C.

Generally, crystalline Form G of almotriptan can be obtained by at least crystallizing almotriptan free base in one or more solvents. Almotriptan free base can be prepared, for example, by reacting the mesylate of 5-(1-pyrrolidinyl methane sulfonyl)-1H-indole-3-ethanol with an aqueous dimethylamine solution.

Suitable solvents for use in the process of the present invention include, but are not limited to, esters of carboxylic acids, aromatic hydrocarbons, aliphatic hydrocarbons, ketones, aromatic alcohols, aliphatic alcohols and the like and mixtures thereof.

Useful esters of carboxylic acids include, but are not limited to, ethyl acetate, isopropyl acetate and the like and mixtures thereof.

Useful aromatic hydrocarbons include, but are not limited to, toluene, xylene, and the like and mixtures thereof.

Useful aliphatic hydrocarbons include, but are not limited to, hexane, heptane and the like and mixtures thereof.

Useful aromatic alcohols include, but are not limited to, C₅-C₃₀ alcohols such as, for example, benzyl alcohol and the like and mixtures thereof. Useful aliphatic alcohols include, but are not limited to, C₁-C₈ alcohols such as, for example, methanol, ethanol, n-propanol, isopropanol, n-butanol, isobutanol, tert-butanol and the like and mixtures thereof.

Ketones for use herein can be ketones having from 3 to about 12 carbon atoms. Representative examples include, but are not limited to, acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone (methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and the like and mixtures thereof.

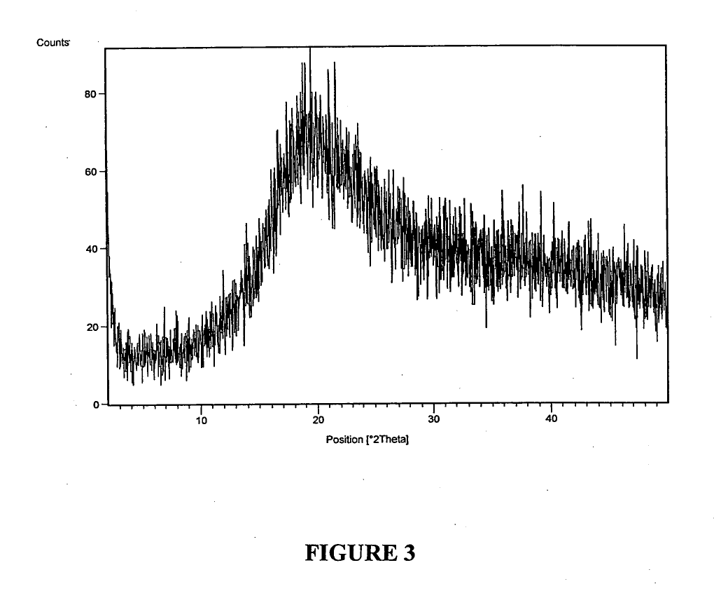

Another embodiment of the present invention is directed to almotriptan malate in an amorphous form. Generally, amorphous materials refer to solid compounds having no substantial crystal lattice structure. Amorphous materials do not exhibit the three dimensional long range order found in crystalline materials, but are structurally more similar to liquids where the arrangement of molecules is random. Substantially amorphous almotriptan malate may contain a very low content of crystalline almotriptan malate, e.g., less than about 5% crystallinity, preferably less than about 2%, and more preferably less than about 1% crystallinity. Preferably, amorphous almotriptan malate is free or substantially free of crystalline almotriptan malate. Crystallinity may be measured as discussed above. The XRD pattern of an amorphous form of almotriptan malate is substantially in accordance with FIG. 3.

Generally, almotriptan malate in an amorphous form can be prepared by at least (a) dissolving substantially non-amorphous almotriptan malate in a solvent solution containing at least one or more solvents capable of dissolving substantially non-amorphous almotriptan malate; and (b) recovering almotriptan malate substantially in an amorphous form from the solution.

In step (a) of the process of the present invention, substantially non-amorphous almotriptan malate is dissolved in a solvent capable of dissolving almotriptan malate to provide a clear solution. The substantially non-amorphous almotriptan malate used as a starting material in the process is well known and can be, for example, hydrates, solvates and the like as well as mixtures of amorphous and non-amorphous forms of the malate salt. In one embodiment, substantially non-amorphous almotriptan malate can be prepared by adding a first solution containing at least an amorphous or crystalline almotriptan base in a first solvent such as an alcoholic solvent; to a second solution containing at least malic acid in a second solvent such as an alcoholic solvent to provide the non-amorphous form of almotriptan malate.

Suitable solvents capable of dissolving substantially non-amorphous almotriptan malate for use herein include, but are not limited to, water, organic solvents, e.g., lower alcohols and the like, and mixtures thereof. Suitable alcohol-containing solvents include aromatic and aliphatic C₁-C₁₂ alcohols and the like and mixtures thereof. Suitable aliphatic alcohols include C₁-C₈ alcohols such as, for example, methanol, ethanol, n-propanol, isopropanol, n-butanol, isobutanol, tert-butanol and the like and mixtures thereof. Suitable aromatic alcohols include C₃-C₁₂ alcohols such as, for example, benzyl alcohol, benzyloxyethanol, phenoxyethanol and the like and mixtures thereof. Preferably the solvent is water or mixtures of water and an alcohol. Generally, the solvent can be present in an amount sufficient to dissolve the starting material, e.g., an amount ranging from about 5 to about 20 and preferably from about 10 to about 20 w/v. The dissolution can be carried out at a temperature ordinarily ranging from about 20° C. to about 80° C. and preferably at room temperature.

If desired, the clear solution of step (a) can be filtered to remove any extraneous matter present in the solution using any standard filtration techniques known in the art. A filtering aid such as celite can be added to the solution to assist in the filtration of the extraneous matter.

In step (b) of the process of the present invention, almotriptan malate in an amorphous form is recovered from the solution. For example, almotriptan malate in an amorphous form is recovered from the solution by substantially removing the solvent from the solution to provide amorphous almotriptan malate as, for example, a free-flowing powder. The solvent may be removed by techniques well known in the art, for example, substantially complete evaporation of the solvent, concentrating the solution, cooling to a temperature sufficient to precipitate an amorphous form and filtering the solid under nitrogen atmosphere. In one embodiment, almotriptan malate in an amorphous form is recovered by spray drying the solution.

When removing the solvent by evaporation, evaporation can be achieved at sub-zero temperatures by the lyophilisation or freeze-drying technique. The solution may also be completely evaporated in a pilot plant Rota vapor, a Vacuum Paddle Dryer or in a conventional reactor under vacuum above about 720 mm Hg by flash evaporation techniques at a temperature of about 90° C., using an agitated thin film dryer (“ATFD”), or evaporated by spray drying at a temperature ranging from about room temperature to about 90° C. to obtain a dry amorphous powder. When the solvent is water, the solvent may be removed by distillation under vacuum in a pilot plant Rota vapor.

In another embodiment, the solution may be concentrated under vacuum, for example, about 720 mm Hg. For example, the initial mass is concentrated to about 1 to about 3 volumes. The concentration may also take place in, for example, a reactor with stirring, a rota vapor or vacuum paddle dryer with stirring. After concentrating the solution, the solution can then be cooled to a temperature of about 0C to obtain a slurry. The slurry can then be filtered under controlled conditions using standard filtration techniques such as over a Nutsche filter, Agitated Nutsche filter, centrifugation, through a filter press or in a sparkler filter. Filtration can typically be carried out under controlled conditions such as, for example, a nitrogen atmosphere, a temperature of about 25° C. and a relative humidity ranging from about 45% to about 50%. The wet product may then be dried. Drying may be accomplished by evaporation, spray drying, drying under vacuum, or freeze-drying. In one embodiment, the wet product can be dried at a temperature of about 60° C.

The amorphous almotriptan malate obtained by the above process may be further dried in, for example, Vacuum Tray Dryer, Rotocon Vacuum Dryer, Vacuum Paddle Dryer or pilot plant Rota vapor to further lower the content of the residual solvents.

By performing this processes of the present invention, substantially pure amorphous almotriptan malate can be prepared with a degree of purity greater than about 95%, preferably greater than about 97% and most preferably greater than about 99.75% as determined by HPLC.

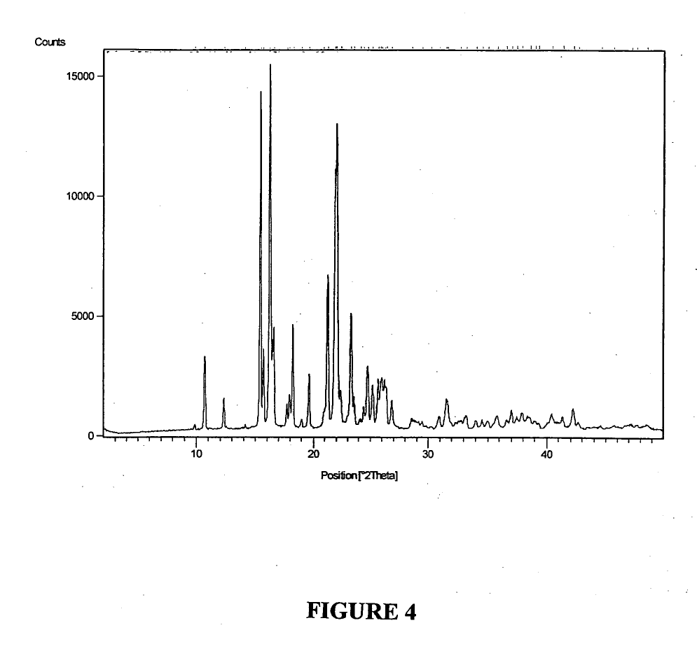

Yet another embodiment of the present invention is directed to a crystalline polymorph of almotriptan malate and having X-ray diffraction (XRD) pattern substantially in accordance with FIG. 4.

In one embodiment, a crystalline form of almotriptan malate can be obtained by (a) reacting almotriptan base with malic acid in a solvent solution comprising a first alcohol; (b) drying the solution to obtain a residue; (c) dissolving the residue in a second alcohol under reflux; and (d) cooling the solution to recover the crystalline form of almotriptan malate from the solution.

In another embodiment, a crystalline form of almotriptan malate can be obtained by (a) providing a solution comprising crystalline Form G of almotriptan and malic acid in a solvent; and (b) substantially removing the solvent from the solution to provide the crystalline form of almotriptan malate.

Solvents can be selected from aliphatic alcohols include, but are not limited to, C₅-C₃₀ alcohols such as, for example, benzyl alcohol and the like and mixtures thereof. Useful aliphatic alcohols include, but are not limited to, C₁-C₈ alcohols such as, for example, methanol, ethanol, n-propanol, isopropanol, n-butanol, isobutanol, tert-butanol and the like and mixtures thereof.

Yet another embodiment of the present invention is directed to pharmaceutical compositions containing at least a therapeutically effective amount of one or more of the crystalline Form G of almotriptan, crystalline almotriptan malate and amorphous almotriptan malate of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. Crystalline Form G of almotriptan, crystalline almotriptan malate and amorphous almotriptan malate of the present invention may also be administered as suppositories, ophthalmic ointments and suspensions, and parenteral suspensions, which are administered by other routes. The dosage forms may contain crystalline Form G of almotriptan, crystalline almotriptan malate and amorphous almotriptan malate of the present invention as is or, alternatively, as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain crystalline Form G of almotriptan, crystalline almotriptan malate and amorphous almotriptan malate of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

In one embodiment, crystalline Form G of almotriptan, crystalline almotriptan malate and amorphous almotriptan malate of the present invention for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. It is to be understood that each of crystalline Form G of almotriptan, crystalline almotriptan malate and amorphous almotriptan malate of the present invention can be of varying particle sizes. It is noted the notation Dₓ means that X % of the particles have a diameter less than a specified diameter D. Thus, a D₅₀ of about 400 microns means that 50% of the micronized particles in a composition have a diameter less than about 400 microns. The term “micronization” used herein means any process or methods by which the size of the particles is reduced. For example, the particle sizes of crystalline Form G of almotriptan, crystalline almotriptan malate and amorphous almotriptan malate of the present invention can be obtained by any milling, grinding, micronizing or other particle size reduction method known in the art to bring the solid state forms into any of the foregoing desired particle size range.

Actual dosage levels of crystalline Form G of almotriptan, crystalline almotriptan malate and amorphous almotriptan malate of the present invention may be varied to obtain an amount that is effective to obtain a desired therapeutic response for a particular composition and method of administration. The selected dosage level therefore depends upon such factors as, for example, the desired therapeutic effect, the route of administration, the desired duration of treatment, and other factors. The total daily dose of the compounds of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. The pharmaceutical compositions herein can formulated in any release form, e.g., immediate release, sustained release, controlled release, etc.

The following examples are provided to enable one skilled in the art to practice the invention and are merely illustrative of the invention. The examples should not be read as limiting the scope of the invention as defined in the features and advantages.

### Experimental

The purity was measured by high performance liquid chromatography (HPLC) under the following conditions:
Column: Column: Inertsil ODS-3V, 250×4.6 mm, 5μ
Moving phase: Mobile Phase A: Dilute 0.56 ml of o-phosphoric acid in 1000ml of water.
Adjust pH to 7.5 with triethylamine.

Mobile Phase B=Acetonitrile

Gradient:

| Time | % Mobile Phase A | % Mobile Phase B |
|---|---|---|
| 0.01 | 10 | 90 |
| 20 | 50 | 50 |
| 30 | 75 | 25 |
| 40 | 75 | 25 |
| 45 | 10 | 90 |
| 50 | 10 | 90 |

Diluent: Water:Acetonitrile (50:50, v/v)
Detector: UV, 227 nm
Flow rate: 1.0 ml/minute
Retention time: 15.0 minutes
Injection volume: 10 μl

### EXAMPLE 1

Preparation of Non-amorphous Almotriptan Malate

Almotriptan base (1.0 kg) was dissolved in denatured spirit (10 L) at room temperature (about 25° C. to about 30° C.). A malic acid solution containing malic acid (0.4 kg) dissolved in methanol (4 L) was added slowly to the solution of the almotriptan base under nitrogen atmosphere. The reaction mixture was stirred for about 1 hour and filtered under nitrogen atmosphere. It was washed with denatured spirit or rectified spirit or absolute alcohol (1.0 L). The wet cake was dried at about 60° C. The resulted material was dissolved in methanol (4 L) and reflux under stirring for 45 minutes followed by cooling at 25 to 30° C. The resulted mass was filtered and dried at 50 to 55° C. under vacuum.
Weight: 1.0 kg.

### EXAMPLE 2

Preparation of Crystalline Form G of Almotriptan

Methanesulfonyl chloride (2.9 g) was added to a solution of 5-(1-pyrrolidinyl methane sulfonyl)-1H-indole-3-ethanol (7 g) in tetrahydrofuran (140 ml) and triethylamine (6 g) at a temperature of below about −20° C. The reaction mass was stirred at a temperature of about −20° C. for a period of about 30 minutes. After completion of the reaction as determined by TLC, a dimethyl amine solution (40%, 55 ml) was added at a temperature of about −20° C. After the addition, the temperature of the reaction mass was allowed to rise to room temperature; a temperature ranging from about 25° C. to about 30° C., and the reaction mass was stirred. After completion of the reaction as determined by TLC, tetrahydrofuran was distilled off under vacuum at a temperature below about 40° C. A 25% w/v potassium carbonate solution (10 ml) was added to the residue and the compound was extracted with isopropyl acetate (2×75 ml). The organic layer was combined and concentrated under vacuum to get a residue (5.6 g) which was taken up in methylene dichloride (20 ml) and treated with succinic acid aqueous solution (2 g in 10 ml water) to wash out impurities in the organic layer by retaining the product as its soluble succinate salt in the aqueous layer. The aqueous layer was treated with sodium carbonate solution to adjust the pH to about 9.0 and the free base was then extracted in isopropyl acetate. The solvent was then distilled off under vacuum to get an oil (3 g). The oil was titrated with a mixture of isopropyl acetate (20 ml) and n-heptane (20 ml) to obtain the almotriptan base as a solid. It was then filtered under suction and dried in vacuum oven at 60° C. Weight: 2 g.
HPLC Purity: 99.56%
Melting Point: 102° C.-104° C.

The XRD pattern and DSC thermogram of the almotriptan obtained in the above example is in accordance with FIGS. 1 and 2 and shows the almotriptan is Form G.

### EXAMPLE 3

Preparation of Non-amorphous Almotriptan Malate

Almotriptan base Form G of Example 2 (1.0 kg) was dissolved in absolute alcohol (10 L) at room temperature (about 25° C. to about 30° C.). A malic acid solution containing malic acid (0.4 kg) dissolved in methanol (4 L) was added slowly to the solution of the almotriptan base under nitrogen atmosphere. The reaction mixture was stirred for about 1 hour and filtered under nitrogen atmosphere. It was washed with denatured spirit or rectified spirit or absolute alcohol (1.0 L). The wet cake was dried at about 60° C.
Weight: 1.0 kg.

### EXAMPLE 4

Preparation of Amorphous Almotriptan Malate

The non-amorphous almotriptan malate of Example 1 (1.0 kg) was dissolved in water at room temperature and filtered through a filtration medium or filter aid to remove any extraneous matter. The clear solution was lyophilized (freeze dried) for 24 hours to provide a dry free-flowing amorphous powder. Similar results were obtained starting with the non-amorphous almotriptan malate of Example 3.
Weight: 1.0 kg.

### EXAMPLE 5

Preparation of Amorphous Almotriptan Malate

The non-amorphous almotriptan malate of Example 1 (1.0 kg) was dissolved in water at room temperature and filtered through a filtration medium or filter aid to remove any extraneous matter. The clear solution was spray-dried in a ‘Lab-plant’ model spray drier at about 90° C. to provide a dry free-flowing amorphous powder.
Weight: 0.7 kg.

### EXAMPLE 6

Preparation of Amorphous Almotriptan Malate

The non-amorphous almotriptan malate of Example 1 (1.0 kg) was dissolved in water at room temperature and filtered through a filtration medium or filter aid to remove any extraneous matter. The clear solution was subjected to distillation in a pilot plant Rota vapor under high vacuum until a dry free-flowing amorphous powder is obtained. Similar results may be obtained when starting with the non-amorphous almotriptan malate of Example 3.
Weight: 1.0 kg.

### EXAMPLE 7

Preparation of Amorphous Almotriptan Malate

Almotriptan base (1.0 kg) was dissolved in methanol (5 L) at room temperature. A malic acid solution containing malic acid (0.4 kg) dissolved in water (5 L) was added slowly to the solution of the almotriptan base under nitrogen atmosphere. The reaction mixture was stirred for 1 hour. Methanol was stripped off under vacuum completely and the resulting solution was filtered to remove any extraneous matter. The clear solution was subjected to lyophilisation for 24 hours until a free flowing amorphous solid was obtained. Similar results may be obtained when starting with the non-amorphous almotriptan malate of Example 3.
Weight: 1.0 kg.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. Crystalline Form G of almotriptan.

2. The crystalline Form G of almotriptan of claim 1, characterized by a powder x-ray diffraction (XRD) pattern having characteristic peaks (expressed in degrees 2θ±0.2°θ) at one or more of the following positions: about 13.96, about 14.25, about 15.48, and about 20.25.

3. The crystalline Form G of almotriptan of claim 1, characterized by having a predominant endotherm peak at about 102.79° C.

4. The crystalline Form G of almotriptan of claim 1, characterized by having at least one of the following characteristics:
(a) a powder XRD pattern substantially in accordance with FIG. 1; and/or
(b) a Differential Scanning Calorimetric (DSC) thermogram substantially in accordance with FIG. 2.

5. The crystalline Form G of almotriptan of claim 1, having a purity of equal to or greater than about 99.5%.

6. A pharmaceutical composition comprising a therapeutically effective amount of the crystalline Form G of almotriptan of claim 1, and one or more pharmaceutically acceptable excipients.

7. The pharmaceutical composition of claim 6, wherein the crystalline Form G of almotriptan is a micronized crystalline Form G of almotriptan having a particle size of less than about 15 microns.

8. A process for preparing crystalline Form G of almotriptan, the process comprising crystallizing almotriptan free base in one or more solvents.

9. The process of claim 8, wherein the solvent is selected from the group consisting of an ester of a carboxylic acid, aromatic hydrocarbon, aliphatic hydrocarbon, ketone, aromatic alcohol, aliphatic alcohol and mixtures thereof.

10. The process of claim 8, wherein the solvent is a mixture of isopropyl acetate and n-heptane.

11. An amorphous form of almotriptan malate.

12. The amorphous form of almotriptan malate of claim 11, having a powder XRD pattern substantially in accordance with FIG. 3.

13. The amorphous almotriptan malate of claim 11, containing less than about 5% of any crystalline form of almotriptan malate

14. The amorphous almotriptan malate of claim 11, having a purity equal to or greater than about 99%.

15. A pharmaceutical composition comprising a therapeutically effective amount of the amorphous almotriptan malate of claim 11, and one or more pharmaceutically acceptable excipients.

16. The pharmaceutical composition of claim 15, wherein the amorphous almotriptan malate is a micronized amorphous almotriptan malate having a particle size of less than about 15 microns.

17. A process for preparing an amorphous form of almotriptan malate, the process comprising the steps of:
(a) dissolving substantially non-amorphous almotriptan malate in a solvent solution comprising one or more solvents capable of dissolving substantially non-amorphous almotriptan malate; and
(b) recovering the amorphous form of almotriptan malate from the solution.

18. The process of claim 17, wherein the solvent is selected from the group consisting of water, alcohol and mixtures thereof.

19. The process of claim 17, wherein the step of recovering comprises distilling the solvent from the solution.

20. The process of claim 17, wherein the step of recovering comprises lyophilizing the solution.

21. The process of claim 17, wherein the step of recovering comprises spray drying.

22. The process of claim 17, wherein the amorphous almotriptan malate recovered in step (b) is substantially pure.

23. A process for preparing an amorphous form of almotriptan malate, the process comprising the steps of:
(a) reacting almotriptan base with malic acid in a solvent solution comprising an alcohol; and
(b) lyophilizing the solution to form the an amorphous form of almotriptan malate.

24. A crystalline form of almotriptan malate.

25. The crystalline form almotriptan malate of claim 24, characterized by having an XRD pattern substantially in accordance with FIG. 4.

26. A pharmaceutical composition comprising a therapeutically effective amount of the crystalline form of almotriptan malate of claim 24, and one or more pharmaceutically acceptable excipients.

27. A process for preparing the crystalline form of almotriptan malate of claim 24, the process comprising the steps of:
(a) reacting almotriptan base with malic acid in a solvent solution comprising a first alcohol; and
(b) drying the solution to obtain a residue;
(c) dissolving the residue in a second alcohol under reflux; and
(d) cooling the solution to recover the crystalline form of almotriptan malate from the solution.

28. A process for preparing the crystalline form of almotriptan malate of claim 24, the process comprising (a) providing a solution comprising crystalline Form G of almotriptan and malic acid in a solvent; and (b) substantially removing the solvent from the solution to provide the crystalline form of almotriptan malate.
